SEQUENCE LISTING (14 sequences)

SEQ ID NO: 1 - protein, 488 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 364 aa, Homo sapiens
MGMPMGSLQPLATLYLLGMLVASCLGRLRVPQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 393 aa, Homo sapiens
MGMPMGSLQPLATLYLLGMLVASCLGRLRVPYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDTWGTYLLTTETGLQKDHALWDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 382 aa, Homo sapiens
MGMPMGSLQPLATLYLLGMLVASCLGRLRVPLWVSQPLEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 434 aa, Homo sapiens [sequence omitted]

SEQ ID NO: 6 - protein, 326 aa, Homo sapiens
MGMPMGSLQPLATLYLLGMLVASCLGRLRVPSPASASTQTSWTPRDLVSSQTQTQSCVPPTAGARQAPESPSTIPVPSQPQNSTLRPGPAAPDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 376 aa, Homo sapiens
MGMPMGSFQPLATLYLLGMLVASCLGRLRVPQSKAQVLQSVAGQTLTVRCQYPPTGSLYEKKGWCKEASALVCIRLVTSSKPRTVAWTSRFTIWDDPDAGFFTVTMTDLREEDSGHYWCRIYRPSDNSVSKSVRFYLVVSPADPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 1506 bp, Homo sapiens
tccccactgctcagcacttaggccggcagaatctgagcgatgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccaggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 9 - DNA, 1110 bp, Homo sapiens
aagcttgccgccaccatgggaatgcccatggggtctctgcaaccgctggccaccttgtacctgctggggatgctggtcgcttcctgcctcggacggctcagggtaccccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - DNA, 1197 bp, Homo sapiens
aagcttgccgccaccatgggaatgcccatggggtctctgcaaccgctggccaccttgtacctgctggggatgctggtcgcttcctgcctcggacggctcagggtaccctatgacacacccaccctctcggttcatcctggacccgaggtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgacacttggggcacctaccttttaaccacagagacgggactccagaaagaccatgccctctgggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 11 - DNA, 1164 bp, Homo sapiens
aagcttgccgccaccatgggaatgcccatggggtctctgcaaccgctggccaccttgtacctgctggggatgctggtcgcttcctgcctcggacggctcagggtacccctctgggtgtcccagccccttgagattcgtaccctggaagggtcttctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - DNA, 1320 bp, Homo sapiens
aagcttgccgccaccatgggaatgcccatggggtctctgcaaccgctggccaccttgtacctgctggggatgctggtcgcttcctgcctcggacggctcagggtaccccaatccaaggctcaggtacttcaaagtgtggcagggcagacgctaaccgtgagatgccagtacccgcccacgggcagtctctacgagaagaaaggctggtgtaaggaggcttcagcacttgtgtgcatcaggttagtcaccagctccaagcccaggacggtggcttggacctctcgattcacaatctgggacgaccctgatgctggcttcttcactgtcaccatgactgatctgagagaggaagactcaggacattactggtgtagaatctaccgcccttctgacaactctgtctctaagtccgtcagattctatctggtggtatctccagcctctgcctccacacagacctcctggactccccgcgacctggtctcttcacagacccagacccagagctgtgtgcctcccactgcaggagccagacaagcccctgagtctccatctaccatccctgtcccttcacagccacagaactccacgctccgccctggccctgcagccccggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 13 - DNA, 996 bp, Homo sapiens
aagcttgccgccaccatgggaatgcccatggggtctctgcaaccgctggccaccttgtacctgctggggatgctggtcgcttcctgcctcggacggctcagggtaccctctccagcctctgcctccacacagacctcctggactccccgcgacctggtctcttcacagacccagacccagagctgtgtgcctcccactgcaggagccagacaagcccctgagtctccatctaccatccctgtcccttcacagccacagaactccacgctccgccctggccctgcagccccggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - DNA, 1146 bp, Homo sapiens
aagcttgccgccaccatgggaatgcccatggggtctctgcaaccgctggccaccttgtacctgctggggatgctggtcgcttcctgcctcggacggctcagggtaccccaatccaaggctcaggtacttcaaagtgtggcagggcagacgctaaccgtgagatgccagtacccgcccacgggcagtctctacgagaagaaaggctggtgtaaggaggcttcagcacttgtgtgcatcaggttagtcaccagctccaagcccaggacggtggcttggacctctcgattcacaatctgggacgaccctgatgctggcttcttcactgtcaccatgactgatctgagagaggaagactcaggacattactggtgtagaatctaccgcccttctgacaactctgtctctaagtccgtcagattctatctggtggtatctccagcggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2010/0047169 A1
(43) Pub. Date: Feb. 25, 2010

(54) NK Cell Receptor Conjugates for Treating Malignancies

(75) Inventors: Ofer MANDELBOIM, Shoam (IL); Angel PORGADOR, Lehavim (IL)

Correspondence Address: WINSTON & STRAWN LLP;PATENT DEPARTMENT, 1700 K STREET, N.W., WASHINGTON, DC, 20006, US

(73) Assignee: NATSPEARS, LTD., Ramat Gan (IL)

(21) Appl. No.: 12/574,478

(22) Filed: Oct. 6, 2009

Related U.S. Application Data
(63) Continuation of application No. 10/538,231, filed on Nov. 25, 2005.
(60) Provisional application No. 60/431,728, filed on Dec. 9, 2002.

(51) Int. Cl. A61K 51/08; A61P 35/00; A61K 38/16; A61K 39/00

(52) U.S. Cl. 424/1.69; 514/12; 424/178.1

(57) ABSTRACT

The present invention relates generally to compositions useful in therapies involving the selective destruction of tumor cells in vivo. In particular, this invention relates to a conjugate which comprises a target recognition segment and an active cytotoxic segment. The target recognition segment comprises a receptor specific to NK cells, wherein the receptor binds to a cellular ligand expressed on the surface of a tumor cell, and the active segment comprises an agent capable of exerting a cytotoxic effect on the tumor cell. The target recognition segment derived form the natural killer receptor NKp30 has been found to be particularly effective in vivo.

### FIELD OF THE INVENTION

The present invention relates generally to compositions useful in the treatment of various cancers and to therapies involving the selective destruction of tumor cells in vivo. More specifically, the present invention relates to conjugates and fusion proteins of Natural Killer cytotoxicity receptors NKp30, NKp46 and NKp44, or active fragments thereof and an active agent selected from a cytotoxic drug or an Ig fragment effective in targeting tumor cells in vivo. Particularly preferred compositions are conjugates and fusion proteins of the NK cell specific receptor NKp30 with the Fc fragment of an Ig molecule.

### BACKGROUND OF THE INVENTION

A key to the development of successful antitumor agents is the ability to design agents that will kill tumor cells selectively, while exerting relatively little, if any, untoward effects against normal tissues. This goal has been elusive to achieve, in that there are few qualitative differences between neoplastic and normal tissues. Because of this, much research over the years has focused on identifying tumor-specific antigens that can serve as immunological targets both for chemotherapy and diagnosis. Many tumor-specific, or quasi-tumor-specific (“tumor-associated”), markers have been identified as tumor cell antigens that can be recognized by specific antibodies. Unfortunately, it is generally the case that tumor-specific antibodies of themselves will not exert sufficient antitumor effects to make them useful in cancer therapy.

Natural Killer (NK) cells destroy virus-infected and transformed cells apparently without prior antigen stimulation (1, 2). The interaction between NK cells and their targets is mediated via a complex array of NK inhibitory and activating receptors (3-7). Heavily implicated in this interplay are inhibitory receptors of the NK cell surface, the ligands of which are polymorphic and non-polymorphic major histocompatibility complex (MHC) class I molecules (3-7). With regard to activation, some NK cells express activation receptors specific for MHC class I molecules homologous to various NK inhibitory receptors (3-7).

Three novel lysis receptors, expressed mainly on human NK cells, were recently identified. These natural cytotoxicity receptors (NCR) include the NKp30, NKp44, and NKp46 molecules (3, 5). All of these NCR are capable of mediating direct killing of tumor and virus-infected cells and are specific for non-MHC ligands. NKp46 and NKp30 are present exclusively on NK cells, whether resting or activated, while NKp44 is expressed specifically by activated NK cells (3, 5).

The most distinctive role of the NCRs in NK cells activity has been attributed to their involvement in recognition and killing of tumor cells. This has become evident by the ability of anti-NCR monoclonal antibodies to block NK-mediated killing of most tumor lines (8-11) and by the strict correlation that exists between the density of NCRs expression on NK cells and their ability to kill tumor targets (11). More recently, the importance of NCRs in vivo was illustrated in acute myeloid leukemia (AML) patients expressing insufficient amount of either NCR or NCR ligands, thereby rendering the leukemia cells resistant to NK cytotoxicity (12).

International Patent Application WO 02/08287 of the present inventors discloses NK receptor fusion proteins in which the extracellular portion of the various NK receptors is conjugated to an active segment selected from an immunoglobulin (Ig) or Ig fragment, a cytotoxic moiety or an imaging moiety. WO 02/08287 further discloses that the NK receptor fusion proteins exhibit specific interactions with tumor cells and viral-infected cells, and these fusion proteins are disclosed as useful for therapeutic applications ex vivo, as well as in vivo. WO 02/08287 generally discloses fusion proteins comprising any one of the three NK cytotoxicity receptors, namely NKp46, NKp44 and NKp30, Specific fusion proteins are disclosed and claimed only for NKp46 and NKp44. The teachings of WO 02/08287 are incorporated herein as if set forth herein in their entirety. It is to be understood explicitly that the present invention excludes any of the specific compositions claimed in WO 02/08287.

WO 01/36630 discloses NKp30, and the use of NKp30 for detecting NK cells and for selective removal of NK cells from a sample. WO 01/36630 further discloses the use of NKp30 antiserum for in vitro stimulation of NK cells cytotoxicity. WO 01/36630 neither discloses nor teaches any therapeutic utility of NKp30 against malignant diseases or even specific targeting of a tumor cell in vivo using either NKp30 or NKp30 fusion proteins.

There exists a long-felt need for anti-tumor agents that will be selective for tumor cells, while exerting relatively little, if any, untoward effects against normal tissues.

### SUMMARY OF THE INVENTION

It is an object of the present invention to provide compositions and methods for inhibiting or reducing the growth of a tumor in a patient. It is a further object of the present invention to provide compositions and therapeutic methods for selectively lysing tumor cells or for selectively delivering a cytotoxic drug to a target tumor cell in a patient for the purpose of eliminating the target tumor cell either directly or by creating an environment lethal to the target cell.

The present invention relates generally to Natural Killer Cytotoxicity receptor (NCR) conjugates and fusion proteins that are effective in targeting tumor cells in vivo. The present invention further relates to NCR conjugates and fusion proteins that are effective in inhibiting the growth of the tumor or reducing the size of the tumor.

The NCR conjugates or fusion proteins according to the present invention comprise a first segment comprising an NCR selected from the group consisting of: NKp46, NKp44 and NKp30, or active fragments thereof, and a second segment comprising an agent active in inducing cytotoxicity. The conjugates or fusion proteins according to the invention comprise an active segment selected from a cytotoxic substance, an Immunoglobulin (Ig) molecule or an active fragment of an Ig molecule such as the Fe fragment of Ig. It is to be understood explicitly that the present invention discloses and claims certain novel conjugates and fusion proteins, particularly those comprising NKp30 or active fragments thereof.

Unexpectedly, it is now disclosed that of the various NK conjugates and fusion proteins tested, those comprising the natural killer cytotoxicity receptor NKp30 or active fragments thereof were found to be especially effective in inhibiting the growth of tumors in vivo. As exemplified herein below by way of a non-limiting example, NKp30 conjugates according to the present invention were particularly effective in inhibiting growth of PC3 prostate tumor cells in vivo as compared to NKp46 and NKp44 conjugates.

According to one aspect, the present invention provides a polypeptide conjugate and pharmaceutical compositions comprising same, wherein the polypeptide conjugate comprises as a first segment an NCR or fragments thereof and as a second segment an agent active in inducing cytotoxicity.

According to one embodiment, the present invention provides a polypeptide conjugate comprising a first segment selected from the group consisting of: NKp30, NKp44, NKp46 or a functional fragments thereof; and a second segment selected from the group consisting of: an Ig molecule, a fragment of an Ig molecule and an Fc portion of an Ig molecule.

According to a preferred embodiment, the polypeptide conjugate comprises NKp30 or a functional fragment thereof and an Fc portion of an Ig molecule.

According to a currently preferred embodiment, the polypeptide conjugate comprises the amino acid sequence set forth herein as SEQ ID NO:4.

According to yet another embodiment, the present invention provides a pharmaceutical composition comprising as an active ingredient a conjugate capable of eliminating a tumor or inhibiting the growth of the tumor in a subject, and a pharmaceutically acceptable carrier, the conjugate comprising:
(a) a target recognition segment comprising an NCR or an active fragment thereof, the NCR selected from the group consisting of: NKp30, NKp46, and NKp44, or a functional fragment thereof, wherein the NCR binds to a cellular ligand expressed on the surface of a target tumor cell;
(b) an active segment comprising a cytotoxic agent that promotes the lysis of the target tumor cell; and
(c) a pharmaceutically acceptable carrier or diluent.

According to one preferred embodiment of the invention the pharmaceutical composition comprises as an active ingredient a polypeptide conjugate comprising NKp30 or a functional fragment thereof and an Fc portion of an Ig molecule.

According to a currently preferred embodiment, the pharmaceutical composition comprises as an active ingredient a polypeptide conjugate having the sequence set forth herein as SEQ ID NO:4.

According to another aspect, the present invention provides isolated polynucleotides encoding the polypeptide conjugates of the present invention, constructs comprising the polynucleotides, vectors comprising these constructs, hosts cells harboring these vectors, and means of producing the polypeptides from cultures of these host cells.

According to one embodiment, the present invention provides an isolated polynucleotide sequence encoding the polypeptide conjugate of the invention.

According to another embodiment, the isolated polynucleotide encodes a polypeptide conjugate comprising NKp30 or a functional fragment thereof and an Fe portion of an Ig molecule.

According to one preferred embodiment, the polynucleotide encodes a polypeptide having the sequence set forth herein as SEQ ID NO:4.

According to a currently preferred embodiment, the polynucleotide comprises the sequence set forth herein as SEQ ID NO:11, or a functional fragment thereof.

According to yet another embodiment, the present invention provides a vector comprising a polynucleotide encoding a polypeptide conjugate comprising NKp30 or a functional fragment thereof and an Fc portion of an Ig molecule.

According to yet another embodiment, the vector is a plasmid or a virus. According to yet another embodiment, the vector is a virus selected from the group consisting of: adenoviruses, retroviruses and lentiviruses.

According to yet another embodiment, the vector further comprises at least one regulatory element operably linked to the polypeptide conjugate of the invention, the at least one regulatory element is selected from the group consisting of: promoter, initiation codon, stop codon, polyadenylation signal, enhancer and selection marker.

According to one embodiment, the present invention provides a host cell comprising the vector of the invention. According to another embodiment the host cell is eukaryotic or prokaryotic. According to yet another embodiment, the present invention provides a host cell capable of expressing the polypeptide conjugate of the invention.

According to yet another aspect, the present invention provides a method for treating a malignant disease in a subject comprising administering to the subject a pharmaceutically effective amount of a polypeptide conjugate comprising:
a) a target recognition segment comprising a Natural Killer cytotoxic receptor (NCR) or an active fragment thereof, wherein the NCR is selected from the group consisting of: NKp46, NKp44, NKp30 or a functional fragment thereof, that binds to a cellular ligand expressed on the surface of a target tumor cell; and
b) an active segment comprising an active agent, the active agent being capable of exerting a cytotoxic effect on said target cell,
   the conjugate being capable of eliminating or inhibiting the growth of the tumor cells associated with the disease, thereby treating the disease.

According to a currently preferred embodiment of the invention the polypeptide conjugate used in this method comprises NKp30 and an Fc portion of an Ig molecule having the sequence set forth herein as SEQ ID NO:4.

According to yet another aspect, the present invention provides a method of inhibiting the growth of a tumor in a subject comprising administering to the subject an effective amount of a polypeptide conjugate comprising:
a) a target recognition segment comprising an NCR or an active fragment thereof, wherein said NCR is selected from the group consisting of: NKp46, NKp44, NKp30 or a functional fragment thereof, wherein the target recognition segment is capable of binding to a cellular ligand expressed on the surface of a target tumor cell; and

b) an active segment comprising an active agent that promotes the lysis of the target tumor cell, thereby inhibiting the growth of the tumor in said subject.

According to a currently preferred embodiment of the invention the polypeptide conjugate used in this method comprises NKp30 and an Fc portion of an Ig molecule having the sequence set forth herein as SEQ ID NO:4.

It is noted that in certain cases, the conjugates of the present invention are capable of eliminating the tumor to the extent that there is no gross evidence for the presence of a tumor in the subject. In other cases, the conjugates of the present invention are capable of reducing the size of the tumor significantly. Preferably the conjugates are capable of reducing the size of a solid tumor by 50% of the initial size, more preferably to 30% of the initial size, most preferably to 10% of the initial size. The size of the tumor in the subject may be determined by any of the diagnostic or imaging techniques as are well known in the art, including for example by Computed Tomography Imaging (CT) or Magnetic Resonance Imaging (MRI).

The NCR conjugates according to the present invention comprise an NCR selected from the group consisting of: NKp30, NKp46 and NKp44, or active fragments thereof. The conjugates according to the invention further comprise an active segment that is selected from a cytotoxic substance, an Immunoglobulin (Ig) molecule or an active fragment of the Ig including but not limited to the Fc fragment of Ig.

According to one exemplary embodiment, the NCR conjugate comprises NKp46 covalently attached to the Fc portion of an Ig molecule, the amino acid sequence of which is denoted as SEQ ID NO:1 and the nucleotide sequence of which is denoted as SEQ ID NO:8. In a preferred embodiment, the NCR conjugate comprises the domain 2 (D2 domain) of NKp46 covalently attached to the Fc molecule, the amino acid sequence of which is denoted as SEQ ID NO:3 and the nucleotide sequence of which is denoted as SEQ ID NO:10. It is noted that while both Isoform A and Isoform B of NKp46 may be used in the conjugates of the invention, the A Isoform is preferred.

According to one currently more preferred embodiment, the NCR conjugate comprises NKp30 covalently attached to the Fc portion of an Ig molecule, the amino acid sequence of which is set forth herein as SEQ ID NO:4 and the nucleotide sequence of which is set forth herein as SEQ ID NO:11. This NKp30-Fc conjugate was unexpectedly discovered to be very effective in inhibiting the growth of a tumor in vivo. It is to be understood explicitly that any analog, derivative or other conjugate having the same attributes is encompassed within the scope of the present invention.

According to yet another embodiment, the NCR conjugate comprises NKp44 covalently attached to Fc molecule, the amino acid sequence of which is denoted as SEQ ID NO:5 and the nucleotide sequence of which is denoted as SEQ ID NO:12.

According to yet another embodiment, the NCR conjugate comprises the NK44-DS fragment covalently attached to Fc molecule, the amino acid sequence of which is denoted as SEQ ID NO:6 and the nucleotide sequence of which is denoted as SEQ ID NO:13. In yet another embodiment, the NCR conjugate comprises the NK44-DL fragment covalently attached to Fc molecule, the amino acid sequence of which is denoted as SEQ ID NO:7 and the nucleotide sequence of which is denoted as SEQ ID NO:14.

As noted above, certain preferred conjugates according to the present invention comprise the Fc fragment of an immunoglobulin molecule as the active segment. The Fc conjugates to the natural killer cytotoxicity receptors is therapeutically useful irrespective of any specific mechanism of action. Without wishing to be bound by any theory, the binding of the Fc-containing conjugates to the target tumor cell can potentially induce cell lysis via three possible mechanisms:

1) Cellular Cytotoxicity—Several different leukocytes including neutrophils, eosonophils, macrophages and NK cells contain Fc receptors and are capable of binding to conjugate-bound tumor cells via the Fc segment, resulting in the lysis of the tumor cells.
2) Complement-Dependent Cytotoxicity—The lysis of the conjugate-bound target tumor cells may be mediated by activation of the complement pathway.
3) Induction of programmed cell death (Apoptosis) following the binding of the Fc-containing conjugates to the target tumor cells and activation of apoptosis intracellular signaling pathways leading to cell death.

According to yet another aspect, the present invention provides a method of delivering a cytotoxic substance to a target tumor cell in a subject in need thereof comprising:

b. administering to the subject a therapeutically effective amount of a conjugate comprising a first segment being a target recognition segment comprising an NCR or an active fragment thereof, the NCR selected from the group consisting of NKp46, NKp44, NKp30 or a functional fragment thereof, and a second segment comprising a cytotoxic agent, the target recognition segment capable of binding to a cellular ligand expressed on the surface of said target tumor cell, wherein the binding of the conjugate to the cellular ligand promotes the internalization of said conjugate into said target tumor cell, thereby delivering said cytotoxic agent to said target tumor cell.

According to one preferred embodiment, the conjugate for delivering a cytotoxic substance to a target tumor cell in a subject comprises NKp30 or an active fragment thereof. According to additional preferred embodiments, the conjugate for delivering a cytotoxic substance to a target tumor cell in a subject comprises NKp46 or domain 2 of NKp46 covalently linked to a cytotoxic substance. The advantage of the conjugates of the present invention is that the binding of the conjugate to the cellular ligand promotes the internalization of said conjugate within said target tumor cell. Thus, it is preferred that the conjugates comprise a cytotoxic agent which promotes cell death upon internalization of the cytotoxic agent into the cell. Preferred cytotoxic agents include, but are not limited to, radioisotopes, steroids, chemotherapeutic drugs, and antisense oligonucleotides.

According to one embodiment, the chemotherapeutic agents are selected from the group consisting of: a hormone such as a steroid; an antimetabolite such as cytosine arabinoside, fluorouracil, methotrexate or aminopterin; an anthracycline; mitomycin C; a vinca alkaloid; demecolcine; etoposide; mithramycin; an antitumor alkylating agent such as chlorambucil. Other drugs such as busulfan, carmustine, cisplatin, cyclophsphamide, doxorubicin, ifosfamide, nitrogen mustards, nitrosureas, melphalan or antitumor antibiotics such as bleomycin or daunorubicin may be used.

According to another embodiment, the radioisotope agents for therapeutic use may be for example: iodine¹³¹, iodine¹²³, technecium^{99m}, indium¹¹¹, rhenium¹⁸⁸, rhenium¹⁸⁶, galium⁶⁷, copper⁶⁷, yttrium⁹⁰, iodine¹²⁵ or astatine²¹¹.

According to another embodiment, the cytotoxic substance may be a cytotoxin such as a plant-, fungus- or bacteria-derived toxin. For example, a ribosome inactivating protein, α-sarcin, aspergillin, restrictocin, a ribonuclease, diphtheria toxin or pseudomonas exotoxin may be used as a cytotoxic substance.

It is proposed that the various methods and compositions of the present invention will be broadly applicable to the treatment of any tumor, including solid and non-solid tumors. If the neoplastic tissue is a part of the lymphatic or immune systems, the non-solid tumors may include circulating malignant cells. Malignancies of other tissues or organs may produce solid tumors. In one preferred example, the conjugates according to the present invention are useful in the treatment of a neoplastic disease associated with solid tumors including but not limited to prostate cancer, melanoma, colon cancer, breast cancer, pancreatic cancer, ovarian cancer, osteosarcoma and renal cell carcinoma.

These and further embodiments will be apparent from the detailed description and examples that follow.

### BRIEF DESCRIPTION OF THE DRAWINGS

FIG. 1 demonstrates the binding of NKp46, NKp44, or NKp30 conjugates to various cancer cells but not to normal peripheral blood lymphocytes (PBL).

FIG. 2 demonstrates the binding of the NKp46-D1-Ig and NKp46-D2-Ig conjugates to melanoma cell lines and prostate cancer cell lines.

FIG. 3 demonstrates the killing of human prostate cancer cells by NKp30-Ig and NKp46D2-Ig.

FIG. 4 demonstrates NKp30-Ig-mediated tumor regression in nude mice.

FIG. 5 illustrates the complete or partial response obtained by the NKp30-Ig conjugate as revealed by the size of the tumor.

FIG. 6 demonstrates that NKp46D2-Ig conjugate binds to the cell surface of 1106mel cells and then internalized to the intracellular domain.

FIG. 7 demonstrates a strong binding of NKp46D2-Ig and NKp30-Ig to a tissue section derived from adenomacarcinoma but not to a tissue section derived from benign prostatic hyperplasia (BPH).

FIG. 8 demonstrates that NKp30-Ig and NKp46D2-Ig bind to a tissue section derived from malignant melanoma but not to a tissue section derived from benign nevus tissue.

FIG. 9 demonstrates that NKp46D2-Ig specifically binds to a tissue section derived from pancreatic cancer.

### DETAILED DESCRIPTION OF THE INVENTION

In order that this invention may be better understood, the following terms and definitions are herein provided.

The term “specific binding” as used herein refers to the preferential association of a molecule with a cell or tissue bearing a particular target molecule or marker and not to cells or tissues lacking that target molecule or expressing that target molecule at low levels. It is, of course, recognized that a certain degree of non-specific interaction may occur between a molecule and a non-target cell or tissue.

The term “conjugate” refers to a polypeptide formed by the joining of two or more polypeptides through a peptide bond formed between the amino terminus of one polypeptide and the carboxyl terminus of another polypeptide. The conjugate may be formed by the chemical coupling of the constituent polypeptides or it may be expressed as a single polypeptide fusion protein from a nucleic acid sequence encoding the single contiguous conjugate. In those instances where the active segment of the conjugate is a cytotoxic agent that is not a polypeptide it is to be understood that the cytotoxic agent is attached via chemical coupling to the polypeptide.

The term “cellular ligand” refers generally to tumor cell membrane molecules capable of reacting with the target recognition segment of the conjugate of the invention.

The term “target recognition segment” refers to a targeting segment capable of specifically recognizing and binding to a target tumor cell via its cellular ligand.

The term “delivering a cytotoxic substance to a target tumor cell” means that the amount of substance associated with a target tumor cell is at least two-fold, preferably 5-fold, more preferably 10-fold higher than the amount of the substance associated with a normal cell.

The term “target cells” refers to cells that are killed by the cytotoxic activity of the conjugate of the invention. The target cells express the ligand for at least one of NKp46, NKp44 and NKp30 molecules and include, in particular, cells that are malignant or otherwise derived from solid as well as non-solid tumors.

The terms “NKp30, NKp44, and NKp46” refer to natural cytotoxicity receptors expressed on human NK cells which are capable of mediating direct killing of tumor and virus-infected cells.

The term “active fragments” refers to “fragments”, “variants”, “analogs” or “derivatives” of the molecule. A “fragment” of a molecule, such as any of the nucleic acid or the amino acid sequence of the present invention is meant to refer to any nucleotide or amino acid subset of the molecule. A “variant” of such molecule is meant to refer to a naturally occurring molecule substantially similar to either the entire molecule or a fragment thereof. An “analog” of a molecule is a homologous molecule from the same species or from different species. The amino acid sequence of an analog or derivative may differ from the specific molecule, e.g. the NKp46, NKp30 or NKp44 molecule, used in the present invention when at least one residue is deleted, inserted or substituted.

The term “cytotoxic effect” refers to a killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell.

The term “complement-mediated lysis” refers to the process by which the complement-dependent coagulation cascade is activated, multi-component complexes are assembled, ultimately generating a lytic complex that has direct lytic action, causing cell permeabilization.

The term “cell-mediated cytotoxicity or destruction” refers to antibody-dependent, cell-mediated cytotoxicity (ADCC) and natural killer (NK) cell killing.

The present invention provides a method for treating a subject having a tumor using an NCR conjugate for specific targeting of tumor cells in vivo. The NCR conjugate of the invention comprises a target recognition segment and an active segment. The target recognition segment comprises a receptor specific to NK cells or a fragment thereof, wherein the receptor binds to a cellular ligand expressed on the surface of a tumor cell, and the active segment comprises an active substance, said active substance is capable of exerting a cytotoxic effect on the tumor cell.

The method of the invention relates to a specific elimination of tumor cells in vivo, and comprises administering to a subject at least one dose of a pharmaceutically effective amount of the NCR conjugate of the invention. The “pharmaceutically effective amount” is an amount of the NCR conjugate effective to specifically kill at least a portion, and preferably a significant portion, of the tumor cells, upon binding of the NCR conjugate to a cellular ligand expressed on the surface of said tumor cells.

In one embodiment, the NCR conjugate comprises NKp46 covalently attached to Fc molecule, the amino acid sequence of which is denoted as SEQ ID NO:1 and the nucleotide sequence of which is denoted as SEQ ID NO:8. In a preferred embodiment, the NCR conjugate comprises the domain 2 (D2 domain) of NKp46 covalently attached to the Fc molecule, the amino acid sequence of which is denoted as SEQ ID NO:3 and the nucleotide sequence of which is denoted as SEQ ID NO:10. It is noted that while both Isoform A and Isoform B of NKp46 may be used in tlie conjugates of the invention, the A Isoform is preferred.

In more preferred embodiment, the NCR conjugate comprises NKp30 covalently attached to Fc molecule, the amino acid sequence of which is denoted as SEQ ID NO:4 and the nucleotide sequence of which is denoted as SEQ ID NO:1. The NKp30-Fc conjugate was unexpectedly discovered to be very effective in inhibiting the growth of a tumor in vivo.

In yet another embodiment, the NCR conjugate comprises NKp44 covalently attached to Fc molecule, the amino acid sequence of which is denoted as SEQ ID NO:5 and the nucleotide sequence of which is denoted as SEQ ID NO:12. In yet another embodiment, the NCR conjugate comprises the NK44-DS fragment covalently attached to Fc molecule, the amino acid sequence of which is denoted as SEQ ID NO:6 and the nucleotide sequence of which is denoted as SEQ ID NO:13. In yet another embodiment, the NCR conjugate comprises the NK44-DL fragment covalently attached to Fc molecule, the amino acid sequence of which is denoted as SEQ ID NO:7 and the nucleotide sequence of which is denoted as SEQ ID NO:14.

A further aspect the invention relates to a pharmaceutical composition comprising as an active ingredient a conjugate comprising:
a target recognition segment comprising an NCR or an active fragment thereof, the NCR selected from the group consisting of: NKp46, NKp44, NKp30 or a functional fragment thereof that binds to a cellular ligand expressed on the surface of a target tumor cell; and
an active segment comprising an active agent that promotes the lysis of the target tumor cell;
and a pharmaceutically acceptable carrier, stabilizer or diluent.

The active segment of the composition of the invention may be an Ig fragment, preferably the Fc portion of an Ig molecule. Alternatively, the active segment may be a cytotoxic substance, such as a chemotherapeutic agent, a radiotherapeutic agent or a cytotoxin capable of exerting a cytotoxic effect on the tumor cell.

In preferred embodiments, the method of the invention includes administering to a subject in need a pharmaceutical composition comprising an amount of NCR-Ig conjugate effective to induce specific cytotoxicity in tumor cells or other diseased cells.

Without wishing to be bound by any particular theory or mechanism of action, according to one embodiment the cytotoxicity may be Antigen-Dependent Cellular Cytotoxicity (ADCC). Specifically, the NCR-Ig conjugates can induce the activation of immune cells that are equipped with Fc or complement receptors, such as macrophages and NK cells. These cells are capable of binding to the NCR-Ig conjugate-coated tumor cells and eliminate them via phagocytosis or via cell-mediated cytotoxicity.

According to an alternative embodiment, the NCR-Ig conjugates of the present invention may also activate the complement-mediated lysis of at least a portion of the tumor cells. The complement system is activated principally by the binding of the first classical pathway component, C1, to the Fc portion of antigen-complexed antibody molecules. Therefore, the conjugates comprising NKp30, NKp46 or NKp44, and the Fc portion of an Ig molecule, can serve as target for the complement system in vivo.

In another embodiment, the method of the invention employs a conjugate wherein the NCR is covalently attached to a cytotoxic substance. According to the principles the conjugates of the invention encompass the use of any cytotoxic substance or agent that can be conjugated to the target recognition segment of an NCR, and can be thus targeted or delivered in active form to the target cell. In any event, it is proposed that cytotoxic agents may be successfully conjugated to the targeting segment in a manner that will allow their targeting, internalization, release or presentation to the target cells as required. Conjugation may be accomplished using any known conjugation technologies as are well known in the art [for example, Ghose, et al., Critical Reviews in Therapeutic Drug Carrier Systems, 3:256-359 (1987)].

It is to be appreciated that the targeting segment of the conjugate of the invention may be linked to the active segment, either directly or indirectly by conjugating or coupling these segments to any one of lipid backbone or carbohydrate backbone.

Exemplary cytotoxic substances include chemotherapeutic agents, radioisotopes as well as cytotoxins. In one embodiment, the chemotherapeutic agents are selected from the group consisting of: a hormone such as a steroid; an antimetabolite such as cytosine arabinoside, fluorouracil, methotrexate or aminopterin; an anthracycline; mitomycin C; a vinca alkaloid; demecolcine; etoposide; mithramycin; an antitumor alkylating agent such as chlorambucil. Other drugs such as busulfan, carmustine, cisplatin, cyclophsphamide, doxorubicin, ifosfamide, nitrogen mustards, nitrosureas, melphalan or antitumor antibiotics such as bleomycin or daunorubicin may be used.

In another embodiment, the radioisotope agents for therapeutic use may be for example: iodine¹³¹, iodine¹²³, teclmecium^{99m}, indium¹¹¹, rhenium¹⁸⁸, rhenium¹⁸⁶, galium⁶⁷, copper⁶⁷, yttrium⁹⁰, iodine¹²⁵ or astatine²¹¹.

In another embodiment, the cytotoxic substance may be a cytotoxin such as a plant-, fungus- or bacteria-derived toxin. For example, a ribosome inactivating protein, α-sarcin, aspergillin, restrictocin, a ribonuclease, diphtheria toxin or pseudomonas exotoxin may be used as a cytotoxic substance.

Particularly preferred cytotoxins include Pseudomonas exotoxins, Diphtheria toxins, ricin, abrin, cytotoxic prodrugs, ribonucleases (e.g., Ribonuclease A), and ribozymes. Pseudomonas exotoxin and Dipthteria toxin, doxorubicin and maytansinoids are most preferred. Pseudomonas exotoxin A (PE) is an extremely active monomeric protein (molecular weight 66 kD), secreted by Pseudomonas aeruginosa, which inhibits protein synthesis in eukaryotic cells. Like PE, diphtheria toxin (DT) kills cells by ADP-ribosylating elongation factor 2 thereby inhibiting protein synthesis.

In another embodiment, the cytotoxic moiety is a cytotoxic prodrug. The conjugate molecule bearing the prodrug is contacted with the target tumor cell thereby localizing the prodrug at the tumor site. The prodrug is then contacted with its corresponding conversion enzyme thereby converting the prodrug into its cytotoxic form at the tumor site thereby causing the inhibition of growth or killing of tumor cells. Suitable prodrugs are well known to those of skill in the art and include, for example, etoposide-4′ phosphate or 7-(2′ aminoethyl phosphate)mitomycin which are activated in the presence of alkaline phosphatase (AP) to effect killing of tumor cells. Other prodrugs include the prodrug N-(p-hydroxyphenoxyacetyl)adriamycin which is used in conjunction with penicillin V amidase (PVA) or 5-fluorocytosine which is used in conjunction with cytosine deaminase (CD) (see, e.g., U.S. Pat. No. 4,957,278).

Ricin and abrin are plant derived cytotoxins well known to those of skill in the art. Like Pseudomonas exotoxin and Diphtheria toxin, ricin and abrin can also be linked to a targeting moiety for specific delivery to cell bearing a particular target molecule. Means of joining ricin and abrin to a targeting molecule are well known to those of skill in the art (see, e.g., Pastan et al. Ann. Rev. Biochem., 61: 331-354 (1992), Thrush et al., Ann. Rev. Imm. 14: 49-71 (1996) and references cited therein).

In another embodiment, the cytotoxic moiety may be an antisense oligonucleotide molecule capable of hybridizing to specific ribonucleotide sequence within the cell, thereby inhibiting the expression of the protein encoded by this ribonucleotide sequence. Antisense oligonucleotides have been safely and effectively administered to humans for inhibiting the expression of specific proteins in the cell. It is thus established that oligonucleotides can be useful therapeutic modalities that can be configured to be useful in treatment regimes for treatment of cells, tissues and animals, especially humans. In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

It is proposed that the various methods and compositions of the invention will be broadly applicable to the treatment of any tumor, including solid and non-solid tumors. If the tissue is a part of the lymphatic or immune systems, malignant cells may include non-solid tumors of circulating cells. Malignancies of other tissues or organs may produce solid tumors. Exemplary solid tumors to which the present invention is directed include but are not limited to carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, neuroblastomas, and the like. Exemplary non-solid tumors to which the present invention is directed include but are not limited to B cell Lymphoma, T cell Lymphoma, or Leukemia such as Chronic Myelogenous Leukemia.

In a preferred embodiment, the conjugates of the present invention are synthesized using recombinant DNA methodology. Generally this involves creating a DNA sequence that encodes the conjugate, placing the DNA in an expression cassette under the control of a particular promoter, expressing the protein in a host, isolating the expressed protein and, if required, renaturing the protein.

DNA encoding the conjugates of this invention may be prepared by any suitable method, including, for example, cloning and restriction of appropriate sequences or direct chemical synthesis by methods such as the phosphotriester method of Narang et al. Meth. Enzymol. 68: 90-99 (1979); the phosphodiester method of Brown et al., Meth. Enzymol. 68: 109-151 (1979); the diethylphosphoramidite method of Beaucage et al., Tetra. Lett., 22: 1859-1862 (1981); and the solid support method of U.S. Pat. No. 4,458,066.

Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences. Alternatively, subsequences may be cloned and the appropriate subsequences cleaved using appropriate restriction enzymes. The fragments may then be ligated to produce the desired DNA sequence.

The nucleic acid sequences encoding the conjugates may be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

Once expressed, the recombinant fusion proteins can be purified according to standard procedures of the art, including ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182. Guide to Protein Purification., Academic Press, Inc. N.Y. (1990)). Substantially pure compositions of at least about 90 to 95% homogeneity are preferred, and 98 to 99% or more homogeneity are most preferred for pharmaceutical uses. Once purified, partially or to homogeneity as desired, the polypeptides may then be used therapeutically.

The composition of the invention further comprises a pharmaceutically acceptable diluent or carrier. The compositions according to the invention will in practice normally be administered orally or by injection. As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

For oral administration tablets and capsules may contain conventional excipients, such as binders, for example syrup, sorbitol, or polyvinyl pyrrolidone; fillers, for example lactose, microcrystalline cellulose, corn starch, calcium phosphate or sorbitol; lubricants, for example magnesium stearate, stearic acid, polyethylene glycol or silica; disintegrates, for example potato starch or sodium starch glycolate, or surfactants, such as sodium lauryl sulphate.

Oral liquid preparations can be in the form of for example water or oil suspensions, solutions, emulsions, syrups or elixirs, or can be supplied as a dry product for constitution with water or another suitable vehicle before use.

The conjugate molecules of this invention are useful for parenteral, topical, oral, or local administration, such as by aerosol or transdermally. The pharmaceutical compositions can be administered in a variety of unit dosage forms depending upon the method of administration; for example oral administration include powder, tablets, pills, capsules and lozenges. It is recognized that the pharmaceutical compositions of this invention, when administered orally, must be protected from digestion. This is typically accomplished either by complexing the conjugate with a composition to render it resistant to acidic and enzymatic hydrolysis or by packaging the conjugate in an appropriately resistant carrier such as a liposome. Means of protecting proteins from digestion are well known in the art.

The pharmaceutical compositions of this invention are particularly useful for parenteral administration, such as intravenous administration or administration into a body cavity or lumen of an organ. The compositions for administration will commonly comprise a solution of the conjugate molecule dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of the conjugate in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Actual methods for preparing parenterally administrable compositions will be known or apparent to those skilled in the art and are described in more detail in such publications as Remington's Pharmaceutical Science, 118th ed., Mack Publishing.

In therapeutic applications, compositions are administered to a patient suffering from a disease, in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the proteins of this invention to effectively treat the patient.

One of skill in the art will appreciate that the therapeutic compositions of this invention can be administered directly to the tumor site. Thus, for example, brain tumors (e.g., gliomas) can be treated by administering the therapeutic composition directly to the tumor site (e.g., through a surgically implanted catheter). Where the fluid delivery through the catheter is pressurized, small molecules (e.g. the therapeutic molecules of this invention) will typically infiltrate as much as two to three centimeters beyond the tumor margin. Alternatively, the therapeutic composition can be placed at the target site in a slow release formulation. Such formulations can include, for example, a biocompatible sponge or other inert or resorbable matrix material impregnated with the therapeutic composition, slow dissolving time release capsules or microcapsules, and the like.

Table 1 below summarizes the list of Sequence ID Numbers which appear in the application.

TABLE 1
Sequence listing

| | | amino acid/ |
|---|---|---|
| SEQ ID NO | Description | nucleotide sequence |
| 1 | NKp46-Fc conjugate | Amino acids |
| 2 | NKp46D1-Fc conjugate | Amino acids |
| 3 | NKp46D2-Fc conjugate | Amino acids |
| 4 | NKp30-Fc conjugate | Amino acids |
| 5 | NKp44-Fc conjugate | Amino acids |
| 6 | NKp44DS-Fc conjugate | Amino acids |
| 7 | NKp44DL-Fc conjugate | Amino acids |
| 8 | NKp46-Fc conjugate | Nucleotides |
| 9 | NKp46D1-Fc conjugate | Nucleotides |
| 10 | NKp46D2-Fc conjugate | Nucleotides |
| 11 | NKp30-Fc conjugate | Nucleotides |
| 12 | NKp44-Fc conjugate | Nucleotides |
| 13 | NKp44DS-Fc conjugate | Nucleotides |
| 14 | NKp44DL-Fc conjugate | Nucleotides |

SEQ ID NO: 1

NKp46 (AJ001383 isoform a) protein
MSSTLPALLCVGLCLSQRISAQQQTLPKPFIWAEPHFMVPKEKQVTICCQ
GNYGAVEYQLHFEGSLFAVDRPKPPERINKVKFYIPDMNSRMAGQYSCIY
RVGELWSEPSNLLDLVVTEMYDTPTLSVHPGPEVISGEKVTFYCRLDTAT
SMFLLLKEGRSSHVQRGYGKVQAEFPLGPVTTAHRGTYRCFGSYNNHAWS
FPSEPVKLLVTGDIENTSLAPEDPTFPADTWGTYLLTTETGLQKDHALWD
HTAQDPEPKSSDKTHTCPPCPAPEFEGAPSVFLFPPKPKDTLMISRTPEV
TCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSK
LTVDKSRWQQGNVFSCSVMHEALHNHYTQKSLSLSPGK

Black letters designate origin leader peptide
Underlined letters designate D1 region
Bold letters designate D2 region
Italic letters designate Fc region (234 amino acid)

SEQ ID NO: 2

NKp46-D1 stable amino acid seq.
MGMPMGSLQPLATLYLLGMLVASCLGRLRVPQQQTLPKPFIWAEPHFMVP
KEKQVTICCQGNYGAVEYQLHFEGSLFAVDRPKPPERINKVKFYIPDMNS
RMAGQYSCIYRVGELWSEPSNLLDLVVTEMDPEPKSSDKTHTCPPCPAPE
FEGAPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALH
NHYTQKSLSLSPGK

Black letters designate leader peptide of CD5
Underlined letters designate KpnI site (5 amino acid)
Bold letters designate D1 region
Italic letters designate Fc region (234 amino acid)

SEQ ID NO: 3

NKp46-D2 stable amino acid seq.
MGMPMGSLQPLATLYLLGMLVASCLGRLRVPYDTPTLSVHFGPEVISGEK
VTFYCRLDTATSMFLLLKEGRSSHVQRGYGKVQAEFPLGPVTTAHRGTYR
CFGSYNNHAWSFPSEPVKLLVTGDIENTSLAPEDPTFPDTWGTYLLTTET
GLQKDHALWDPEPKSSDKTHTCPPCPAPEFEGAPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLEPS
RDELTKNQVSLTCLVKGFYPSDIAVEWESNGQPENNYKTTPPVLDSDGSF
FLYSKLTVDKSRWQQGNVFSCSVMHEALHNHYTQKSLSLSPGK

Black letters designate leader peptide of CD5
Underlined letters designate KpnI site (5 amino acid)
Bold letters designate D2 region (128 amino acid)
Italic letters designate Fc region (234 amino acid)

SEQ ID NO: 4

NKp30 stable amino acid seq.
MGMPMGSLQPLATLYLLGMLVASCLGRLRVPLWVSQPLEIRTLEGSSAFL
PCSFNASQGRLAIGSVTWFRDEVVPGKEVRNGTPEFRGRLAPLASSRFLH
DNQAELHIRDVRGHDASIYVCRVEVLGLGVGTGNGTRLVVEKEHPQLGDP
EPKSSDKTHTCPPCPAPEFEGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

Black letters designate leader peptide of CD5
Underlined letters designate KpnI site (5 amino acid)
Bold letters designate NKp30
Italic letters designate Fc region

SEQ ID NO: 5

NKp44 stable amino acid seq.
MGMPMGSLQPLATLYLLGMLVASCLGRLRVPQSKAQVLQSVAGQTLTVRC
QYPPTGSLYEKKGWCKEASALVCIRLVTSSKPRTVAWTSRFTIWDDPDAG
FFTVTMTDLREEDSGHYWCRIYRPSDNSVSKSVRFYLVVSPASASTQTSW
TPRDLVSSQTQTQSCVPPTAQARQAPESPSTIPVPSQPQNSTLRPGPAAP
DPEPKSSDKTHTCPPCPAPEFEGAPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQV
SLTGLVKGFYPSDIAVEWESNGQPENNYKTTPPVLDSDGSFFLYSKLTVD
KSRWQQGNVFSCSVMHEALHNHYTQKSLSLSPGK

Black letters designate leader peptide of CD5
Underlined letters designate KpnI site (5 amino acid)
Bold letters designate NKp44 region
Italic letters designate Fc region (234-amino acid)

SEQ ID NO: 6

NKp44-DS stable amino acid seq.
MGMPMGSLQPLATLYLLGMLVASCLGRLRVPSPASASTQTSWTPRDLVSS
QTQTQSCVPPTAGARQAPESPSTIPVPSQPQNSTLRPGPAAPDPEPKSSD
KTHTCPPCPAFEFEGAPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGN
VFSCSVMHEALHNHYTQKSLSLSPGK

Black letters designate leader peptide of CD5
Underlined letters designate KpnI site (5 amino acid)
Bold letters designate NKp44-DS region
Italic letters designate Fc region (234-amino acid)

SEQ ID NO: 7

NKp44-DL stable amino acid seq
MGMPMGSFQPLATLYLLGMLVASCLGRLRVPQSKAQVLQSVAGQTLTVRC
QYPPTGSLYEKKGWCKEASALVCIRLVTSSKPRTVAWTSRFTIWDDPDAG
FFTVTMTDLREEDSGHYWCRIYRPSDNSVSKSVRFYLVVSPADPEPKSSD
KTHTCPPCPAPEFEGAPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGN
VFSCSVMHEALHNHYTQKSLSLSPGK

Black letters designate leader peptide of CD5
Underlined letters designate KpnI site (5 amino acid)
Bold letters designate NKp44-DL region
Italic letters designate Fc region (234-amino acid)

SEQ ID NO: 8 NKp46 DNA seq. (AJ001383)
TCCCCACTGCTCAGCACTTAGGCCGGCAGAATCTGAGCGATGTCTTCCAC
ACTCCCTGCCCTGCTCTGCGTCGGGCTGTGTCTGAGTCAGAGGATCAGCG
CCCAGCAGCAGACTCTCCCAAAACCGTTCATCTGGGCCGAGCCCCATTTC
ATGGTTCCAAAGGAAAAGCAAGTGACCATCTGTTGCCAGGGAAATTATGG
GGCTGTTGAATACCAGCTGCACTTTGAAGGAAGCCTTTTTGCCGTGGACA
GACCAAAACCCCCTGAGCGGATTAACAAAGTCAAATTCTACATCCCGGAC
ATGAACTCCCGCATGGCAGGGCAATACAGCTGCATCTATCGGGTTGGGGA
GCTCTGGTCAGAGCCCAGCAACTTGCTGGATCTGGTGGTAACAGAAATGT
ATGACACACCCACCCTCTCGGTTCATCCTGGACCCGAAGTGATCTCGGGA
GAGAAGGTGACCTTCTACTGCCGTCTAGACACTGCAACAAGCATGTTCTT
ACTGCTCAAGGAGGGAAGATCCAGCCACGTACAGCGCGGATACGGGAAGG
TCCAGGCGGAGTTCCCCCTGGGCCCTGTGACCACAGCCCACCGAGGGACA
TACCGATGTTTTGGCTCCTATAACAACCATGCCTGGTCTTTCCCCAGTGA
GCCAGTGAAGCTCCTGGTCACAGGCGACATTGAGAACACCAGCCTTGCAC
CTGAAGACCCCACCTTTCCTGCAGACACTTGGGGCACCTACCTTTTAACC
ACAGAGACGGGACTCCAGAAAGACCATGCCCTCTGGGATCACACTGCCCA
GGATCCGGAGCCCAAATCTTCTGACAAAACTCACACATGCCCACCGTGCC
CAGCACCTGAATTCGAGGGTGCACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAGCCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTACAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGT
AAATGA

SEQ ID NO: 9

NKp46-D1 stable DNA seq. in pCDNA3.1
AAGCTT [symbol] ATG [symbol] GAATGCCCATGGGGTCTCTGCAACCGCTGGC
CACCTTGTACCTGCTGGGGATGCTGGTCGCTTCCTGCCTCGGACGGCTCA
GGGTACCCCAGCAGCAGACTCTCCCAAAACCGTTCATCTGGGCCGAGCCC
CATTTCATGGTTCCAAAGGAAAAGCAAGTGACCATCTGTTGCCAGGGAAA
TTATGGGGCTGTTGAATACCAGCTGCACTTTGAAGGAAGCCTTTTTGCCG
TGGACAGACCAAAACCCCCTGAGCGGATTAACAAAGTCAAATTCTACATC
CCGGACATGAACTCCCGCATGGCAGGGCAATACAGCTGCATCTATCGGGT
TGGGGAGCTCTGGTCAGAGCCCAGCAACTTGCTGGATCTGGTGGTAACAG
AAATGGATCCGGAGCCCAAATCTTCTGACAAAACTCACACATGCCCACCG
TGCCCAGCACCTGAATTCGAGGGTGCACCGTCAGTCTTCCTCTTCCCCCC
AAAACCCAAGGACACCCTCATGATCTCCCGGACCCCTGAGGTCACATGCG
TGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCA
GTACAACAGCACGTACCGTGTGGTCAGCGTCCTCACCGTCCTGCACCAGG
ACTGGCTGAATGGCAAGGAGTACAAGTGCAAGGTCTCCAACAAAGCCCTC
CCAGCCCCCATCGAGAAAACCATCTCCAAAGCCAAAGGGCAGCCCCGAGA
GCCACAGGTGTACACCCTGCCCCCATCCCGGGATGAGCTGACCAAGAACC
AGGTCAGCCTGACCTGCCTGGTCAAAGGCTTCTATCCCAGCGACATCGCC
GTGGAGTGGGAGAGCAATGGGCAGCCGGAGAACAACTACAAGACCACGCC
TCCCGTGCTGGACTCCGACGGCTCCTTCTTCCTCTACAGCAAGCTCACCG
TGGACAAGAGCAGGTGGCAGCAGGGGAACGTCTTCTCATGCTCCGTGATG
CATGAGGCTCTGCACAACCACTACACGCAGAAGAGCCTCTCCCTGTCTCC
GGGTAAATGA

Black letters designate HindIII site
Open box designate Kozak seq. (with the G in position +4)
Underlined letters designate Start codon of the CD5 leader peptide
Bold letters designate CD5 leader peptide
Shaded letters designate KpnI site
Underlined letters designate D1 DNA seq.

Italic letters designate Fc DNA seq.

SEQ ID NO: 10

NKp46-D2 stable DNA seq. in pCDNA3.1
AAGCTT [symbol] ATG [symbol] GAATGCCCATGGGGTCTCTGCAACCGCTGGC
CACCTTGTACCTGCTGGGGATGCTGGTCGCTTCCTGCCTCGGACGGCTCA
GGGTACCCTATGACACACCCACCCTCTCGGTTCATCCTGGACCCGAGGTG
ATCTCGGGAGAGAAGGTGACCTTCTACTGCCGTCTAGACACTGCAACAAG
CATGTTCTTACTGCTCAAGGAGGGAAGATCCAGCCACGTACAGCGCGGAT
ACGGGAAGGTCCAGGCGGAGTTCCCCCTGGGCCCTGTGACCACAGCCCAC
CGAGGGACATACCGATGTTTTGGCTCCTATAACAACCATGCCTGGTCTTT
CCCCAGTGAGCCAGTGAAGCTCCTGGTCACAGGCGACATTGAGAACACCA
GCCTTGCACCTGAAGACCCCACCTTTCCTGACACTTGGGGCACCTACCTT
TTAACCACAGAGACGGGACTCCAGAAAGACCATGCCCTCTGGGATCCGGA
GCCCAAATCTTCTGACAAAACTCACACATGCCCACCGTGCCCAGCACCTG
AATTCGAGGGTGCACCGTCAGTCTTCCTCTTCCCCCCAAAACCCAAGGAC
ACCCTCATGATCTCCCGGACCCCTGAGGTCACATGCGTGGTGGTGGACGT
GAGCCACGAAGACCCTGAGGTCAAGTTCAACTGGTACGTGGACGGCGTGG
AGGTGCATAATGCCAAGACAAAGCCGCGGGAGGAGCAGTACAACAGCACG
TACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCG
AGAAAACCATCTCCAAAGCCAAAGGGCAGCCCCGAGAGCCACAGGTGTAC
ACCCTGCCCCCATCCCGGGATGAGCTGACCAAGAACCAGGTCAGCCTGAC
CTGCCTGGTCAAAGGCTTCTATCCCAGCGACATCGCCGTGGAGTGGGAGA
GCAATGGGCAGCCGGAGAACAACTACAAGACCACGCCTCCCGTGCTGGAC
TCCGACGGCTCCTTCTTCCTCTACAGCAAGCTCACCGTGGACAAGAGCAG
GTGGCAGCAGGGGAACGTCTTCTCATGCTCCGTGATGCATGAGGCTCTGC
ACAACCACTACACGCAGAAGAGCCTCTCCCTCTCTCCGGGTAAATGA

Black letters designate HindIII site
Open box designate Kozak seq. (with thec in position+4)
Underlined letters designate Start codon of the CD5 leader peptide
Bold letters designate CD5 leader
Shaded letters designate KpnI site
Underlined letters designate D2 DNA seq.
Italic letters designate Fc DNA seq.

SEQ ID NO: 11

NKp30 stable DNA seq. in pCDNA3.1
AAGCTT [symbol] ATG [symbol] GAATGCCCATGGGGTCTCTGCAACCGCTGGC
CACCTTGTACCTGCTGGGGATGCTGGTCGCTTCCTGCCTCGGACGGCTCA
GGGTACCCCTCTGGGTGTCCCAGCCCCTTGAGATTCGTACCCTGGAAGGG
TCTTCTGCCTTCCTGCCCTGCTCCTTCAATGCCAGCCAAGGGAGACTGGC
CATTGGCTCCGTCACGTGGTTCCGAGATGAGGTGGTTCCAGGGAAGGAGG
TGAGGAATGGAACCCCAGAGTTCAGGGGCCGCCTGGCCCCACTTGCTTCT
TCCCGTTTCCTCCATGACCACCAGGCTGAGCTGCACATCCGGGACGTGCG
AGGCCATGACGCCAGCATCTACGTGTGCAGAGTGGAGGTGCTGGGCCTTG
GTGTCGGGACAGGGAATGGGACTCGGCTGGTGGTGGAGAAAGAACATCCT
CAGCTAGGGGATCCGGAGCCCAAATCTTCTGACAAAACTCACACATGCCC
ACCGTGCCCAGCACCTGAATTCGAGGGTGCACCGTCAGTCTTCCTCTTCC
CCCCAAAACCCAAGGACACCCTCATGATCTCCCGGACCCCTGAGGTCACA
TGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGG
AGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGC
CCTCCCAGCCCCCATCGACAAAACCATCTCCAAAGCCAAAGGGCAGCCCC
GAGAGCCACAGGTGTACACCCTGCCCCCATCCCGGGATGAGCTGACCAAG
AACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCA
CGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGT
GATGCATGAGGCTCTGCACAACCACTACACGCAGAAGAGCCTCTCCCTGT
CTCCGGGTAAATGA

Black letters designate HindIII site
Open box designate Kozak seq. (with the G in position +4)
Underlined letters designate Start codon of the CD5 leader peptide
Bold letters designate CD5 leader
Shaded letters designate KpnI site
Underlined letters designate 30 DNA seq.
Italic letters designate Fc DNA seq.

SEQ ID NO: 12

NKp44 stable DNA seq. in pCDNA3.1
AAGCTT [symbol] ATG [symbol] GAATGCCCATGGGGTCTCTGCAACCGCTGGC
CACCTTGTACCTGCTGGGGATGCTGGTCGCTTCCTGCCTCGGACGGCTCA
GGGTACCCCAATCCAAGGCTCAGGTACTTCAAAGTGTGGCAGGGCAGACG
CTAACCGTGAGATGCCAGTACCCGCCCACGGGCAGTCTCTACGAGAAGAA
AGGCTGGTGTAAGGAGGCTTCAGCACTTGTGTGCATCAGGTTAGTCACCA
GCTCCAAGCCCAGGACGGTGGCTTGGACCTCTCGATTCACAATCTGGGAC
GACCCTGATGCTGGCTTCTTCACTGTCACCATGACTGATCTGAGAGAGGA
AGACTCAGGACATTACTGGTGTAGAATCTACCGCCCTTCTGACAACTCTG
TCTCTAAGTCCGTCAGATTCTATCTGGTGGTATCTCCAGCCTCTGCCTCC
ACACAGACCTCCTGGACTCCCCGCGACCTGGTCTCTTCACAGACCCAGAC
CCAGAGCTGTGTGCCTCCCACTGCAGGAGCCAGACAAGCCCCTGAGTCTC
CATCTACCATCCCTGTCCCTTCACAGCCACAGAACTCCACGCTCCGCCCT
GGCCCTGCAGCCCCGGATCCGGAGCCCAAATCTTCTGACAAAACTCACAC
ATGCCCACCGTGCCCAGCACCTGAATTCGAGGGTGCACCGTCAGTCTTCC
TCTTCCCCCCAAAACCCAAGGACACCCTCATGATCTCCCGGACCCCTGAG
GTCACATGCGTGGTGGTGGACGTGAGCCACGAAGACCCTGAGGTCAAGTT
CAACTGGTACGTGGACGGCGTGGAGGTGCATAATGCCAAGACAAAGCCGC
GGGAGGAGCAGTACAACAGCACGTACCGTCTGGTCAGCGTCCTCACCGTC
CTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGC
AGCCCCGAGAGCCACAGGTGTACACCCTGCCCOCATCCCGGGATGAGCTG
ACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACA
AGACCACGCCTCCCGTGCTGGACTCCGACGGCTCCTTCTTCCTCTACAGC
AAGCTCACCGTGGACAAGAGCAGGTGGCAGCAGGGGAACGTCTTCTCATG
CTCCGTGATGCATGAGGCTCTGCACAACCACTACACGCAGAAGAGCCTCT
CCCTGTCTCCGGGTAAATGA

Black letters designate HindIII site
Open box designate Kozak seq. (with the G in position +4)
Underlined letters designate Start codon of the CD5 leader peptide
Bold letters designate CD5 leader
Shaded letters designate KpnI site
Underlined letters designate NKp44 DNA seq.
Italic letters designate Fc DNA seq.

SEQ ID NO: 13

NKp44-DS stable DNA seq. in pCDNA3.1
AAGCTT [symbol] ATG [symbol] GAATGCCCATGGGGTCTCTGCAACCGCTGGC
CACCTTGTACCTGCTGGGGATGCTGGTCGCTTCCTGCCTCGGACGGCTCA
GGGTACCCTCTCCAGCCTCTGCCTCCACACAGACCTCCTGGACTCCCCGC
GACCTGGTCTCTTCACAGACCCAGACCCAGAGCTGTGTGCCTCCCACTGC
AGGAGCCAGACAAGCCCCTGAGTCTCCATCTACCATCCCTGTCCCTTCAC
AGCCACAGAACTCCACGCTCCGCCCTGGCCCTGCAGCCCCGGATCCGGAG
CCCAAATCTTCTGACAAAACTCACACATGCCCACCGTGCCCAGCACCTGA
ATTCGAGGGTGCACCGTCAGTCTTCCTCTTCCCCCCAAAACCCAAGGACA
CCCTCATGATCTCCCGGACCCCTGAGGTCACATGCGTGGTGGTGGACGTG
AGCCACGAAGACCCTGAGGTCAAGTTCAACTGGTACGTGGACGGCGTGGA
GGTGCATAATGCCAAGACAAAGCCGCGGGAGGAGCAGTACAACAGCACGT
ACCGTGTGGTCAGCGTCCTCACCGTCCTGCACCAGGACTGGCTGAATGGC
AAGGAGTACAAGTGCAAGGTCTCCAACAAAGCCCTCCCAGCCCCCATCGA
GAAAACCATCTCCAAAGCCAAAGGGCAGCCCCGAGAGCCACAGGTGTACA
CCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAG
CAATGGGCAGCCGGAGAACAACTACAAGACCACGCCTCCCGTGCTGGACT
CCGACGGCTCCTTCTTCCTCTACAGCAAGCTCACCGTGGACAAGAGCAGG
TGGCAGCAGGGGAACGTCTTCTCATGCTCCGTGATGCATGAGGCTCTGCA
CAACCACTACACGCAGAAGAGCCTCTCCCTGTCTCCGGGTAAATGA

Black letters designate HindIII site
Open box designate Kozak seq.(with the G in position +4)
Underlined letters designate Start codon of the CD5 leader peptide
Bold letters designate CD5 leader
Shaded letters designate KpnI site
Underlined letters designate NKp44-DS DNA seq.
Italic letters designate Fc DNA seq.

SEQ ID NO: 14

NKp44-DL stable DNA seq. in pCDNA3.1
AAGCTT [symbol] ATG [symbol] GAATGCCCATGGGGTCTCTGCAACCGCTGGC
CACCTTGTACCTGCTGGGGATGCTGGTCGCTTCCTGCCTCGGACGGCTCA
GGGTACCCCAATCCAAGGCTCAGGTACTTCAAAGTGTGGCAGGGCAGACG
CTAACCGTGAGATGCCACTACCCGCCCACGGGCAGTCTCTACGAGAAGAA
AGGCTGGTGTAAGGAGGCTTCAGCACTTGTGTGCATCAGGTTAGTCACCA
GCTCCAAGCCCAGGACGGTGGCTTGGACCTCTCGATTCACAATCTGGGAC
GACCCTGATGCTGGCTTCTTCACTGTCACCATGACTGATCTGAGAGAGGA
AGACTCAGGACATTACTGGTGTAGAATCTACCGCCCTTCTGACAACTCTG
TCTCTAAGTCCGTCAGATTCTATCTGGTGGTATCTCCAGCGGATCCGGAG
CCCAAATCTTCTGACAAAACTCACACATGCCCACCGTGCCCAGCACCTGA
ATTCGAGGGTGCACCGTCAGTCTTCCTCTTCCCCCCAAAACCCAAGGACA
CCCTCATGATCTCCCGGACCCCTGAGGTCACATGCGTGGTGGTGGACGTG
AGCCACGAAGACCCTGAGGTCAAGTTCAACTGGTACGTGGACGGCGTGGA
GGTGCATAATGCCAAGACAAAGCCGCGGGAGGAGCAGTACAACAGCACGT
ACCGTGTGGTCAGCGTCCTCACCGTCCTGCACCAGGACTGGCTGAATGGC
AAGGAGTACAAGTGCAAGGTCTCCAACAAAGCCCTCCCAGCCCCCATCGA
GAAAACCATCTCCAAAGCCAAAGGGCAGCCCCGAGAGCCACAGGTGTACA
CCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAG
CAATGGGCAGCCGGAGAACAACTACAAGACCACGCCTCCCGTGCTGGACT
CCGACGGCTCCTTCTTCCTCTACAGCAAGCTCACCGTGGACAAGAGCAGG
TGGCAGCAGGGGAACGTCTTCTCATGCTCCGTGATGCATGAGGCTCTGCA
CAACCACTACACGCAGAAGAGCCTCTCCCTGTCTCCGGGTAAATGA

Black letters designate HindIII site
Open box designate Kozak seq. (with the G in position +4)
Underlined letters designate Start codon of the CD5 leader peptide
Bold letters designate CD5 leader
Shaded letters designate KpnI site
Underlined letters designate NKp44-DL DNA seq.
Italic letters designate Fc DNA seq.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Experimental Methods

### Preparation of Nkpi-Ig Conjugates

Sequences encoding the extracellular portions of NKp30, NKp44 and NKp46 were amplified by PCR from cDNA isolated from human NK clones. These PCR-generated fragments were cloned into the mammalian expression vector containing the constant region (Hinge+CH2+CH3) of a human IgG1 heavy chain. The Ig-conjugates (termed as NKpi-Ig) were produced by transfection of mammalian cells (e.g. COS-7). Protein dimers were secreted from transfected cells (grown in medium without serum), and were purified from the supernatant using Protein-G columns. Protein-G binds only to Fc portion of the dimers made of Ig heavy chain constant regions; thus only the dimers of NKpi-Ig were purified.

### Preparation of Nkpi-Ig Stable Clones for Constant Expression

Sequences encoding the extracellular portions of NKp30-Ig, NKp44-Ig, NKp46-Ig, NK46D2-Ig and CD99-Ig as a control protein were amplified by PCR from cDNA isolated from human NK clones, using Kozak primer for high expression. These PCR fragments containing Kozak sequence and leader sequence were cloned into pedant 3.1-Ig vector. CHO cells were transfected with these expression vectors and were selected using G418 antibiotic. Selected clones were screened for highest protein production using ELISA. High producer clones were re-cloned and screened again for highest protein production. One clone was adapted for special serum-free medium followed by optimization for growth in large-scale culture using spinner basket-Fibro cell (New Brunswick) or triple flasks. Supernatant were collected daily for purification on protein-G columns using FPLC.

### Analysis of Various Prostate Cancer Cells for NCR Binding

Expression of ligands to NCRs was quantitated by flow cytometry analysis of NKpi-Ig binding to various prostate cancer cells. Cells were incubated with 10 micrograms of each NKpi-Ig followed by incubation with human Fc-specific-(PE)-conjugated goat-anti-human IgG. MFI indicates Median Fluorescence Intensity; MFI numbers were rounded to the nearest whole numbers. Results are representative of two independent experiments.

Testing the therapeutic efficacy of NKpi-Ig on prostate cancer and melanoma growth in vivo

PC-3 human prostate cancer cells were transfected with plasmid encoding the luciferase gene. Nude mice were injected with PC-3-luciferase cells and monitored for tumor growth twice a week. Tumor growth was monitored using whole-body imaging with a charged-coupled device camera. For real time monitoring of tumor development, mice were anesthetized and injected intraperitoneally with 50 mM luciferin (126 mg/Kg). Bioluminescence was monitored 1 min later, using an intensified charged coupled device camera (model U 6173-01; Hamamatusu, Hamamatusu, Japan). After establishment of tumor, NKpi-Ig was injected daily (0.25 mg/mouse/day) and the growth of tumors in treated and mock-treated mice was monitored.

### Example 1

### Binding of NKpi-Ig to Various Cancer Cells but not to Normal Peripheral Blood Lymphocytes (PBL)

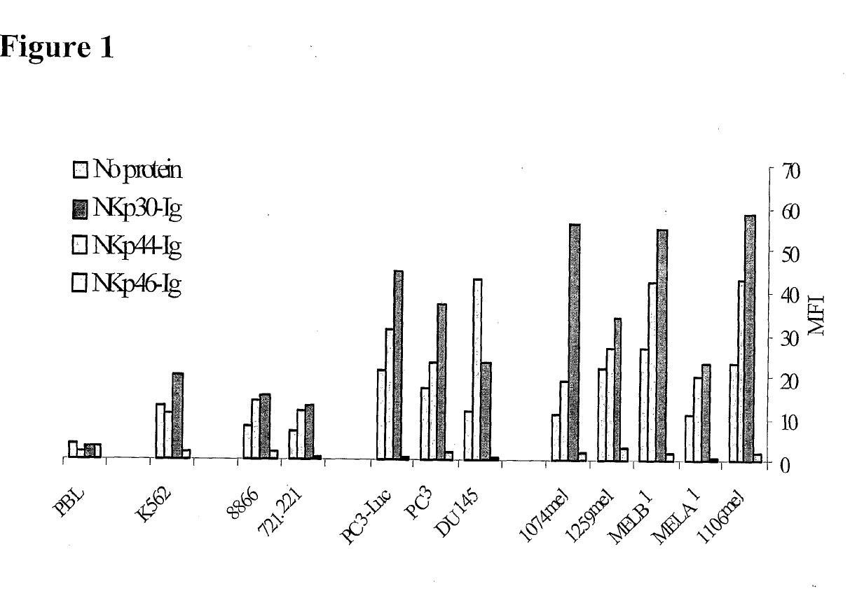

In order to measure the specific NKpi-Ig binding to various tumor cells, cells were incubated with different Ig-conjugates and stained with PE-conjugated goat anti-human Fc. As demonstrated in FIG. 1, different human tumor cell lines (such as cell lines derived from Melanoma (1074mel, 1259mel, MELA1, 1106mel), Chronic Myelogenous Leukemia (CML) (K562), Prostate carcinoma (PC3-Luc, PC3, DU145), weakly EBV-transformed B cells (RPMI8866, 721.221)) were recognized by the various lysis receptors NKp46, NKp44 and NKp30 conjugates. Primary tumor cells were also recognized by the various conjugates, including melanoma and CML cells. No specific NKpi-Ig binding was found in peripheral blood lymphocytes (PBL) normal cells, indicating the specific binding of NKpi-Ig to tumor cells. In addition, T cell lymphoma (Jurkat) and primary breast carcinoma were recognized mainly by NKp30 and NKp44 and to a lesser extent by NKp46.

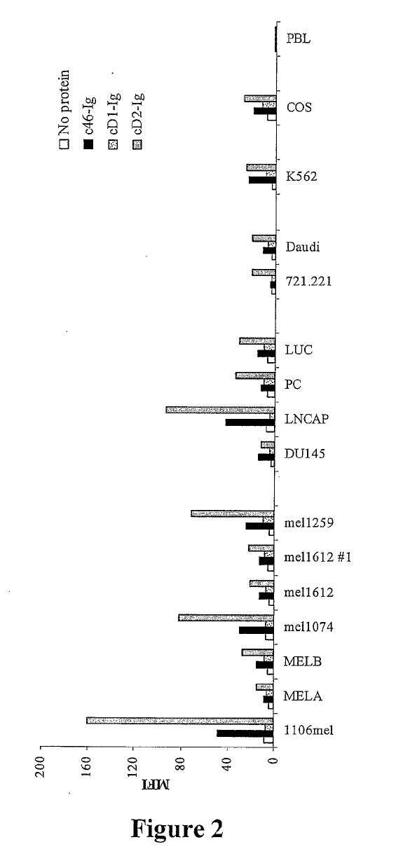

The next step was to test conjugates containing the first domain of NKp46 (NKp46-D1-Ig) or the second domain of NKp46 (NKp46-D2-Ig) in their ability to bind specifically to tumor cells. FIG. 2 demonstrates the binding of the NKp46-D1-Ig and NKp46-D2-Ig conjugates to Melanoma cell lines and prostate cancer cell lines. As can be observed, no significant binding of NKp46-D1-Ig was detected to any of the target cells tested. In contrast, binding of NKp46-D2-Ig was observed to the entire cell lines tested. These findings indicate that NKp46D2-Ig has a better binding capability comparing with NKp46-Ig and NKp30-Ig. Table 2 summarizes the binding profile of NKp30 and NKp46 (30 ug/ml) to various tumor cell lines as determined by FACS analysis. As control for Ig fusion protein CTLA-41 g was used for the carcinomas, in the case of lymphomas E7-Ig was used.

TABLE 2
Binding of NKp30 and NKp46 to tumor cell lines

| Category | Designation | Histological Type | NKp46 | NKp30 |
|---|---|---|---|---|
| Breast | MDA-MB 231 | Adenocarcinoma | Positive | Negative |
| Colon | HCT116 | Adenocarcinoma | Negative | Negative |
| Colon | HT29 | Adenocarcinoma | Positive | Positive |
| Lung | A-549 | Large cell carcinoma | Negative | Negative |
| Prostate | LN-Cap | Metastatic carcinoma, | Positive | Positive |
| | | androgen dependent | | |
| Prostate | PC-3 | Carcinoma, androgen | Positive | Positive |
| | | independent | | |
| B Lymphoma | SKW | EBV positive, B lymphoma | Negative | Negative |
| B Lymphoma | Raji | Burkitt's Lymphoma | Negative | Positive |
| B Lymphoma | Ramos | Burkitt's Lymphoma | Negative | Negative |

### Example 2

### Macrophage-Mediated Antigen-Dependent Cellular Cytotoxicity of tumor Cells with NKp30-Ig and NKp46D2-Ig

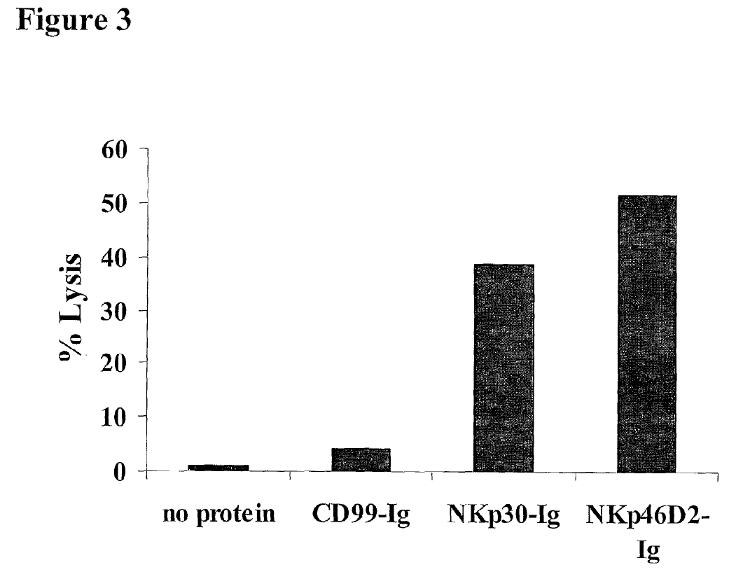

As revealed from FIG. 3, the binding of NKp46D2-Ig and NKp30-Ig conjugates to their unknown ligands on PC3 prostate cancer cells mediates the lysis of the cancer cells via macrophage-dependent lysis mechanism. In vitro killing of PC3 cells coated with the NKp30-Ig or NKp46D2-Ig conjugates by complement and NK cells was not observed.

### Example 3

### In Vivo Tumor Cell Elimination After Treatment with NKp30-Ig

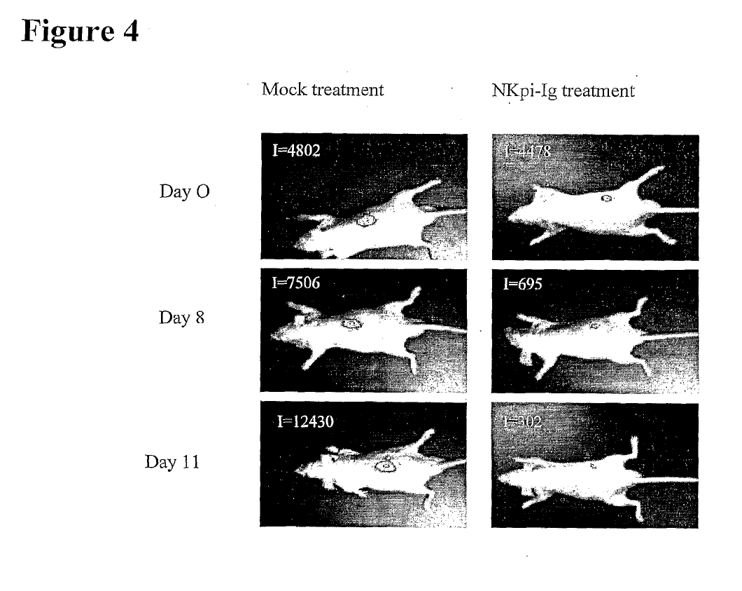

Mice were inoculated S.C. with 2×10⁶ PC-3 human prostate cancer cells transfected with plasmid encoding the luciferase gene. Tumor growth was monitored using whole-body imaging with a charge-coupled device camera. After detectable tumors were established, mice were divided into two groups with similar tumor size distribution. One group received the test conjugate and the other received vehicle control for 3 weeks and the mice were sacrificed. For the test group, NKp30-Ig was injected intraperitoneally daily (0.25 mg/mouse/day) for 3 weeks and the mice were sacrificed. The growth of tumors in treated and mock-treated mice was monitored twice a week. As revealed from FIG. 4 and Table 3, the treatment with NKp30-Ig mediated tumor regression in prostate cancer-bearing nude mice.

TABLE 3
Tumor regression following the treatment with NKp30-Ig.

| | Partial regression | Regression (over | Progressive |
|---|---|---|---|
| Treatment | (less than 50%) | 50%) | growth of tumor |
| Control | 1/6 | 1/6 | 4/6 |
| NKp30-Ig | 1/7 | 5/7 | 1/7 |

Table 4 demonstrates the results following a multiple-dose treatment of NKp30-Ig compared to NKp46D2-Ig on PC3 tumors in nude mice.

TABLE 4
Tumor cell elimination following a multiple-dose treatment of
NKp30-Ig and NKp46D2-Ig

| | Complete | Partial | Progressive |
|---|---|---|---|
| Treatment | response | response | growth of tumor |
| Control | 0/8 | 1/8 | 7/8 |
| NKp30-Ig | 7/17 | 5/17 | 5/7 |
| NKp46D2-Ig | 2/9 | 1/9 | 6/9 |

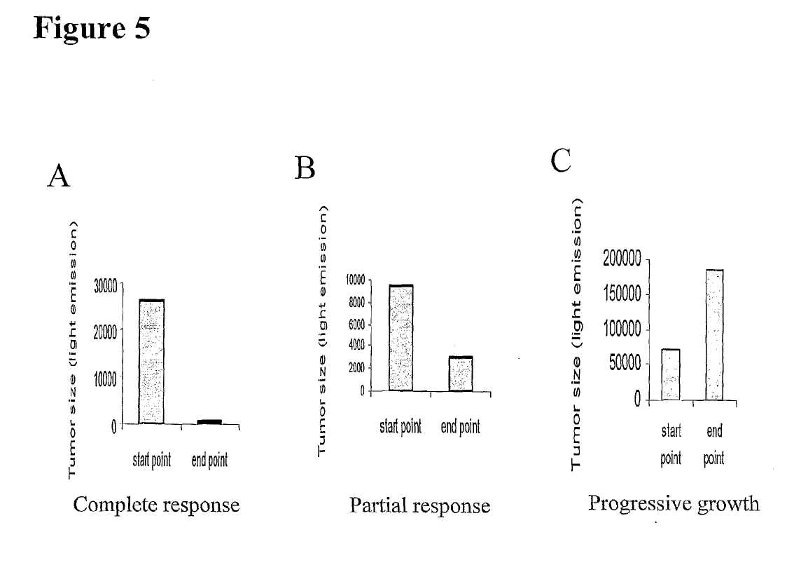

A large-scale experiment was designed to compare the effects of NKp30-Ig and NKp46D2-Ig on growth of tumors in nude mice injected with PC3 prostate cell line. The PC3 cells were injected and three weeks later when tumors were visible, treatments were initiated. As shown in Table 4, the treatment with NKp30-Ig results in complete or partial regression in about two thirds of the mice. FIG. 5 illustrates the response obtained using the NKp30-Ig conjugate as revealed by the size of the tumor. Treatment with NKp46D2-Ig had no discernable effect on tumor growth in this set of experiments (Table 4).

### Example 4

### NKp46D2-Ig Binding is Followed by its Internalization to the Intracellular Domain

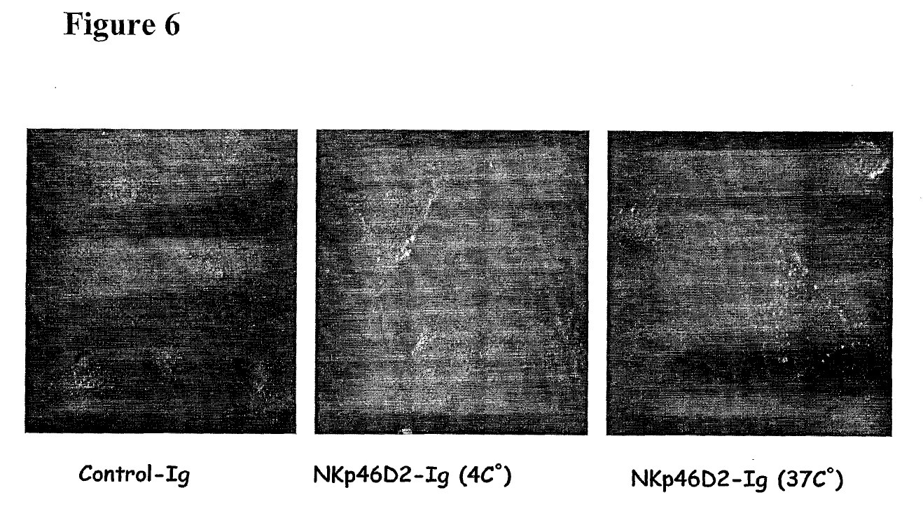

In order to examine whether the NKp46D2-Ig conjugate can be internalized into the target tumor cell, NKp46D2-Ig was incubated with 1106mel and PC3 cell lines followed by fluorescent dye, which can be monitored by confocal microscopy. Conditions suitable for internalization of receptors were applied. FIG. 6 demonstrates that NKp46D2-Ig bind to its ligand on the cell surface of 1106mel cells and then is internalized into the intracellular domain. The same was demonstrated in PC3 cell line, NKp46D2-Ig is located in the intracellular domain after binding to its ligands on PC3 cell line.

### Example 5

### Immunohistochemistry Staining of NKp46D2-Ig and NKp30-Ig Performed on Normal and Tumor Paraffin-Embedded Tissues

Immunohistochemistry staining of NKp46D2-Ig and NKp30-Ig performed on different normal and tumor tissues was conducted. As demonstrated in Table 5, Paraffin-embedded tissues derived from tumors positively react with NKp46D2-Ig and NKp30-Ig. Normal tissues demonstrated negative results in the majority of the examined tissues. Pancreatic islet and chief cells in stomach fundus were the only normal cells that were stained by NKp46D2-Ig.

TABLE 5
Immunohistochemistry staining of NKp46D2-Ig and NKp30-Ig performed
on different normal and tumor paraffin embedded tissues.

| Tissue | Positive |
|---|---|
| Subcutaneous tissue | Angiosarcoma |
| Breast | Ductal carcinoma in situ |
| Breast | Infiltrating duct carcinoma |
| Lymph node | Hodgkin's disease |
| Bone | Osteosarcoma |
| Lung | Adenocarcinoma |
| Liver | Cholangiocarcinoma |
| Liver | Metastatic adenocarcinoma, rectum |
| Duodenum | Stromal tumor, malignant |
| Sigmoid colon | Adenocarcinoma |
| Rectum | Adenocarcinoma |
| Rectum | Mucinous carcinoma |
| Kidney | Renal cell carcinoma |
| Bladder | Transitional cell carcinoma |
| Myometrium | Leiomyoma |
| Ovary | Metastatic mucinous adenocarcinoma, appendix |
| Ovary | Mucinous cyst adenocarcinoma |
| Thyroid | Papillary carcinoma |
| Skin | Malignant melanoma |

Pathologists use the Gleason system to evaluate the differentiation of the adenocarcinoma tissue in the prostate. It is based exclusively on the architectural pattern of the glands of the prostate tumor. It evaluates how effectively the cells of any particular cancer are able to structure themselves into glands resembling those of the normal prostate. The ability of a tumor to mimic normal gland architecture is called its differentiation, and experience has shown that a tumor whose structure is nearly normal (well differentiated) will probably have a biological behavior relatively close to normal—that is not very aggressively malignant. The principle is fairly simple, and Gleason grading from very well differentiated to very poorly differentiated. The lower the Gleason score, the better the patient is likely to survive.

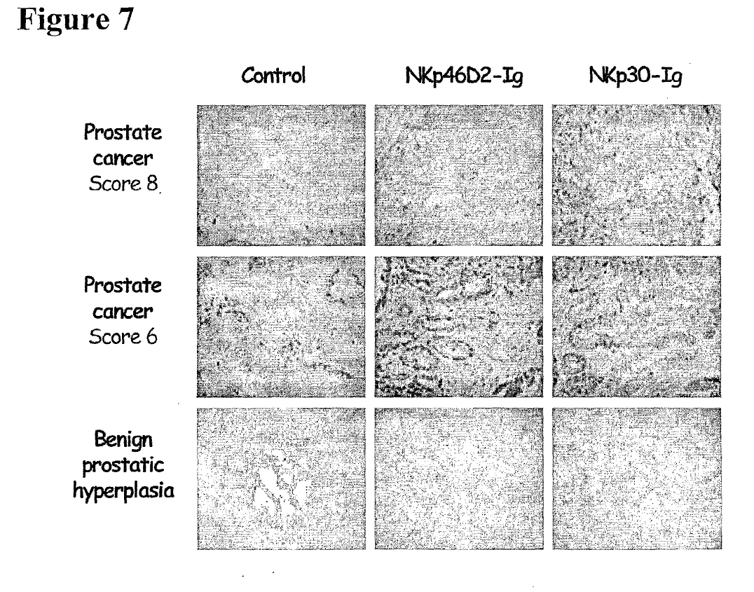

FIG. 7 demonstrates immunohistochemical staining with two prostate adenocarcinomas, one with score 8 and the second with score 6, the last prostate was a benign prostatic hyperplasia (BPH) that in elder man consider to be a “normal” prostate. As revealed from FIG. 7, strong binding of NKp46D2-Ig and NKp30-Ig to the adenocarcinoma and not to the BPH was observed. It is important to note that when the BPH is in the vicinity of an adenocarcinoma it would be stained with NKp46D2-Ig and NKp30-Ig. These findings are unique because it means that the NKp30-Ig and NKp46D2-Ig conjugates can detect tumor-associated changes in the benign hyperplasia in the prostate.

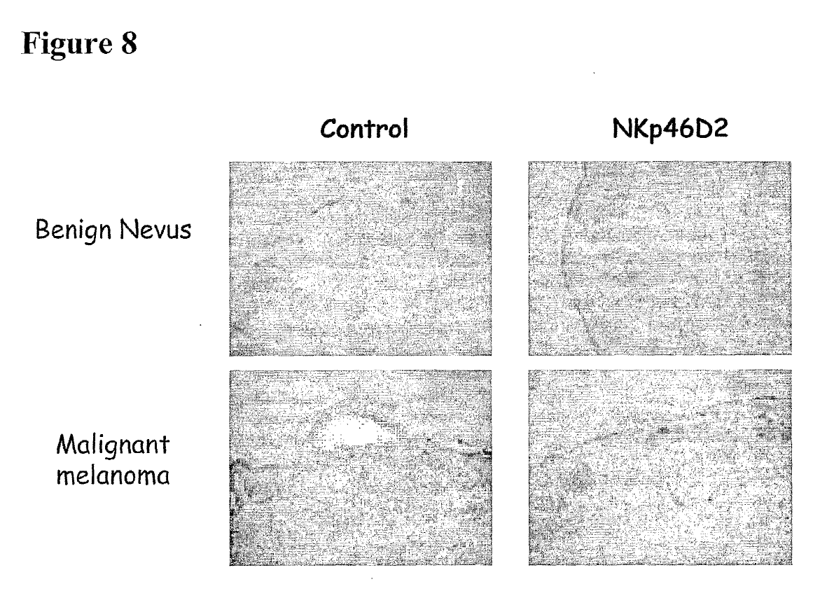

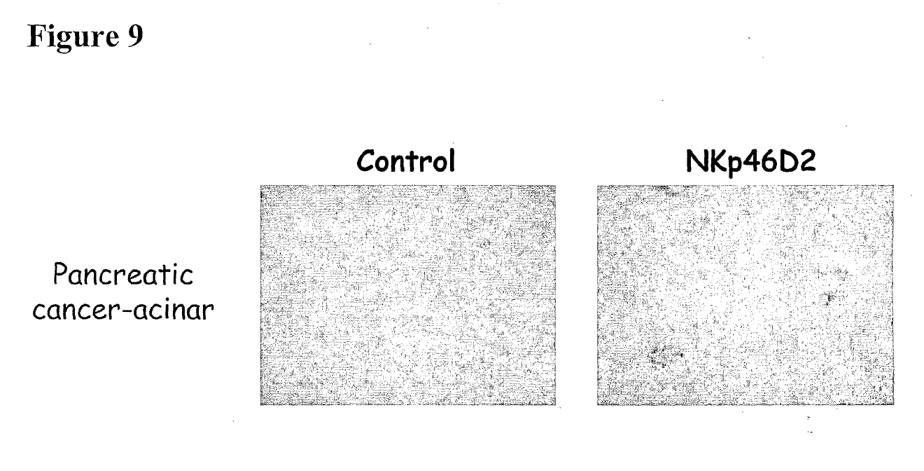

The same pattern was found in melanoma and in pancreatic cancer. As demonstrated in FIG. 8, NKp30-Ig and NKp46D2-Ig bind to malignant melanoma and not to benign nevus which is an early stage of potential melanoma. It is thus another example in which NKp46D2-Ig uniquely and selectively binds to the malignant cancers and to the normal tissues surrounding the tumor and not to benign lesions. Similarly, FIG. 9 demonstrates that NKp46D2-Ig specifically recognizes the tissue derived from pancreatic cancer, one of the most lethal cancers in humans. NKp46D2-Ig binds specifically to pancreatic cancer cells that originate from acinar cells in the pancreas.

### REFERENCES

1. Trinchieri, G. 1989. Biology of natural killer cells. Adv Immunol 47:187-376.
2. Long, E. O. 2002. Tumor cell recognition by natural killer cells. Semin Cancer Biol 12:57-61.
3. Biassoni, R., C. Caiitoni, D. Pende, S. Sivori, S. Parolini, M. Vitale, C. Bottino, and A. Moretta. 2001. Human natural killer cell receptors and co-receptors. Immunol Rev 181:203-214.
4. Bakker, A. B., J. Wu, J. H. Phillips, and L. L. Lanier. 2000. NK cell activation: distinct stimulatory pathways counterbalancing inhibitory signals. Hum Immunol 61:18-27.
5. Moretta, A., C. Bottino, M. Vitale, D. Pende, C. Cantoni, M. C. Mingari, R. Biassoni, and L. Moretta. 2001. Activating receptors and coreceptors involved in human natural killer cell-mediated cytolysis. Annu Rev Immunol 19:197-223.
6. Ljunggren, H. G., and K. Karre. 1990. In search of the ‘missing self’: MHC molecules and NK cell recognition. Immunol Today 11:237-244.
7. McQueen, K. L., and P. Parham. 2002. Variable receptors controlling activation and inhibition of NK cells. Curr Opin Immunol 14:615-621.
8. Pessino A, Sivori S, Bottino C, Malaspina A, Morelli L, Moretta L, Biassoni R, Moretta A. Molecular cloning of NKp46: a novel member of the immunoglobulin superfamily involved in triggering of natural cytotoxicity. J Exp Med. 1998; 188:953-960.
9. Vitale M, Bottino C, Sivori S, Sanseverino L, Castriconi R, Marcenaro E, Augugliaro R, Moretta L, Moretta A. NKp44, a novel triggering surface molecule specifically expressed by activated natural killer cells, is involved in non-major histocoinpatibility complex-restricted tumor cell lysis. J Exp Med. 1998; 187:2065-2072.
10. Cantoni C, Bottino C, Vitale M, Pessino A, Augugliaro R, Malaspina A, Parolini S, Moretta L, Moretta A, Biassoni R. NKp44, a triggering receptor involved in tumor cell lysis by activated human natural killer cells, is a novel member of the immunoglobulin superfamily. J Exp Med. 1999; 189:787-796.
11. Sivori S, Pende D, Bottino C, Marcenaro E, Pessino A, Biassoni R, Moretta L, Moretta A. NKp46 is the major triggering receptor involved in the natural cytotoxicity of fresh or cultured human NK cells. Correlation between surface density of NKp46 and natural cytotoxicity against autologous, allogeneic or xenogeneic target cells. Eur J. Immunol. 1999; 29:1656-1666.
12. Costello R T, Sivori S, Marcenaro E, Lafage-Pochitaloff M, Mozziconacci M J, Reviron D, Gastaut J A, Pende D, Olive D, Moretta A. Defective expression and function of natural killer cell-triggering receptors in patients with acute myeloid leukemia. Blood. 2002; 99:3661-3667.

It will be appreciated by a person skilled in the art that the present invention is not limited by what has been particularly shown and described hereinabove. Rather, the scope of the invention is defined by the claims that follow.

1. A method for treating a malignant disease comprising administering to a subject in need thereof a therapeutically effective amount of a conjugate comprising: (a) a target recognition segment comprising a Natural Killer cell receptor (NCR), wherein the NCR is selected from the group consisting of: NKp30 and a fragment thereof that binds to the surface of a target tumor cell; and (b) a second segment comprising an active agent, wherein the active agent is selected from the group consisting of a cytotoxic agent, an immunoglobulin (Ig) molecule, and a fragment of an Ig molecule; the conjugate being capable of eliminating or inhibiting the growth of the tumor cells associated with the disease; thereby treating the disease.

2. The method according to claim 1, wherein the conjugate is a fusion protein.

3. The method according to claim 2, wherein the fusion protein comprises the amino acid sequence as set forth in SEQ ID NO: 4.

4. The method according to claim 1, wherein the malignant disease is associated with a solid tumor or a non-solid tumor.

5. The method according to claim 1, wherein the cytotoxic agent is selected from the group consisting of: chemotherapeutic agents, radioisotopes and cytotoxins.

6. The method according to claim 1, wherein the fragment of an Ig molecule is the Fc fragment of an Ig molecule.

7. The method according to claim 1, wherein the conjugate comprises NKp30 covalently attached to the Fc fragment of an Ig molecule.

8. The method according to claim 7, wherein the conjugate has the amino acid sequence as set forth in SEQ ID NO: 4.

9. The method according to claim 5, wherein the cytotoxin is a plant-, a fungus- or a bacteria-derived toxin.

10. A method of inhibiting the growth of a tumor comprising administering to a subject in need thereof a therapeutically effective amount of a polypeptide conjugate comprising: (a) a target recognition segment comprising an NCR, wherein said NCR is selected from the group consisting of: NKp30 and a fragment thereof that binds to the surface of a target tumor cell; and (b) an active segment comprising an active substance that promotes the lysis of the target tumor cell, wherein the active agent is selected from the group consisting of a cytotoxic agent, an immunoglobulin (Ig) molecule, and a fragment of an Ig molecule; thereby inhibiting the growth of the tumor in said subject.

11. The method according to claim 10, wherein the polypeptide conjugate is a fusion protein.

12. The method according to claim 11, wherein the fusion protein comprises the amino acid sequence as set forth in SEQ ID NO: 4.

13. The method according to claim 10, wherein the cytotoxic agent is selected from the group consisting of: chemotherapeutic agents, radioisotopes and cytotoxins.

14. The method according to claim 10, wherein the fragment of an Ig molecule is the Fc fragment of an Ig molecule.

15. The method according to claim 10, wherein the solid tumor is selected from the group consisting of: carcinoma, squamous cell carcinoma, adenocarcinoma, small cell carcinoma, melanoma, glioma and neuroblastoma.

16. The method according to claim 10, wherein the conjugate comprises NKp30 covalently attached to the Fc fragment of an Ig molecule.

17. The method according to claim 16, wherein the conjugate has the amino acid sequence as set forth in SEQ ID NO: 4.

18. The method according to claim 13, wherein the cytotoxin is a plant-, a fungus- or a bacteria-derived toxin.
